(19) United States
(12) Patent Application Publication
(10) Pub. No.: US 2003/0171434 A1
(43) Pub. Date: Sep. 11, 2003

(54) Branched amino acids

(76) Inventors: Richard Francis William Jackson, Sheffield (GB); Urszula Grabowska, Cambridge (GB)

Correspondence Address: BIRCH STEWART KOLASCH & BIRCH, PO BOX 747, FALLS CHURCH, VA, 22040-0747, US

(21) Appl. No.: 10/276,443

(22) PCT Filed: May 16, 2001
(86) PCT No.: PCT/GB01/02162

(30) Foreign Application Priority Data
May 17, 2000 (MY) PI 2000 2178
Oct. 17, 2000 (GB) 0025386.4

(51) Int. Cl. A61K 31/198; C07C 229/08

(52) U.S. Cl. 514/561; 562/553

(57) ABSTRACT

The invention relates to novel branched amino acids and novel methods for their production. The amino acids are useful in the preparation of non-natural peptides and peptidomimetics, by efficient synthesis methodology allowing good enantiomeric specificity at the alpha carbon. Typically the stereochemistry at the alpha carbon is at least 85%, preferably at least 95%, such as in excess of 99% enantiomerically pure. L-stereochemistry at this location is convenient as most biological interactions will favour this configuration, but the invention also extends to enantiomerically enriched and preferably at least 85%, preferably at least 95% such as at least 99% enantiomerically pure D stereoconfiguration. Compounds of the invention will find utility in the preparation of non-natural peptides and peptidomimetics, such as those used in the exploration of receptor specificity and activity or in peptidomimetic inhibitors of enzyme function. The compounds of the invention are built into such peptides/peptidomimetics using standard peptide chemistry.

### FIELD OF THE INVENTION

This invention relates to novel branched amino acids and novel methods for their production. The amino acids are useful in the preparation of non-natural peptides and peptidomimetics.

### TECHNICAL BACKGROUND

Unnatural analogues of proteinogenic amino acids comprise an important tool in the context of exploring receptor binding and preparing drug-like molecules able to interact with such receptors. For example, proteases, ie enzymes that cleave proteins or polyproteins at distinct sites are widespread in most organisms studied. Proteases recognize defined amino acid sequences adjacent the cleavage site and the elucidation of this interaction is a first step in the design of peptide or peptidomimetic small molecules able to inhibit protease function. A number of therapeutic areas have been addressed by the inhibition of proteases including infection (for example the cysteine protease of hepatitis C virus (HCV) or the aspartyl protease of HIV) and physiological disorders (for example various cancers with matrix metalloproteases and osteopathic disorders with cysteine proteases such as cathepsins K, L and B).

Whether employed in receptor exploration or peptide/peptidomimetic construction it is important that constituent amino acids, natural or non-natural have a defined stereochemistry at the alpha carbon. Typically this will be the L-stereochemistry, but a number of therapeutics also employ specific amino acids with the D-stereochemistry at this location. Accordingly there is a need for efficient synthesis methodology allowing good enantiomeric specificity at the alpha carbon. Traditional amino acid synthesis techniques have been unable to produce non-natural branched amino acids, especially lipophilic amino acids, with the requisite degree of enantiomeric specificity.

The unprotected branched amino acid corresponding to compound 3 below has been isolated by hydrolysis of the peptide antibiotic Longicatenamycin^{5,6} along with the lower and higher homologues but such processes are not feasible for large scale production of pharmaceutical intermediates or research reagents.

### BRIEF DESCRIPTION OF THE INVENTION

In accordance with a first aspect of the invention, there are provided compounds of the formula I:

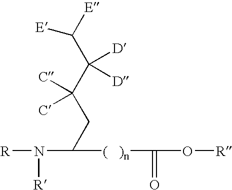

wherein

R is H or an amine protecting group;

R′ is H, C₁-C₆ alkyl, C₂-C₆ alkenyl, ArCₒ-C₆alkyl or HetC₀-C₆alkyl,

R″ is H or a carboxy protecting group;

( ) is a methylene group;

n is 0,1 or2;

C′, C″, D′, E′ and E′ are hydrogen (H) or a group selected from C₁-C₆ alkyl, C₂-C₆ alkenyl, ArC₀-C₆ alkyl or HetC₀-C₆ alkyl, (“Alk”); and

D″ is H or an unsaturation (“ene”) extended between carbon atoms D and E;

in the following permutations:

| C′ | C″ | D′ | D″ | E′ | E″ |
|---|---|---|---|---|---|
| H | H | H | H | Alk | Alk |
| H | H | H | ene | Alk | Alk |
| H | H | Alk | H | Alk | Alk |
| H | H | H | ene | Alk | Alk |
| H | Alk | Alk | H | H | H |
| H | Alk | Alk | ene | H | H |
| Alk | Alk | H | H | H | H |
| Alk | Alk | H | ene | H | H |
| Alk | Alk | Alk | H | H | H |
| Alk | Alk | Alk | ene | H | H |

with the proviso that R, R′ and R″ are not all H when C′, C″ and D′ are all H and E′ and E″ are both methyl.

Typically the stereochemistry at the alpha carbon is at least 85%, preferably at least 95%, such as in excess of 99% enantiomerically pure. L-stereochemistry at this location is convenient as most biological interactions will favour this configuration, but the invention also extends to enantiomerically enriched and preferably at least 85%, preferably at least 95% such as at least 99% enantiomerically pure D stereoconfiguration.

The compounds of the invention comprise gamma (n=2), beta (n=1) or preferably alpha (n=0) amino acids.

The currently preferred values for each occurrence of Alk are C₁-C₆ alkyl, especially C₁-C₃ alkyl, particularly methyl. The Alk for C′, C″, D′, E′ and E″ are chosen independently of each other.

Compounds of the invention will find utility in the preparation of non-natural peptides and peptidomimetics, such as those used in the exploration of receptor specificity and activity or in peptidomimetic inhibitors of enzyme function. The compounds of the invention are built into such peptides/peptidomimetics using standard peptide chemistry.

Elucidating enzyme activity is generally described in Molecular Recognition of Protein-Ligand Complexes: Applications to DrugDesign, Robert E. Babine and Steven L. Bender, Chem. Rev., 1997, 97, 1359-1472 and The therapeutic potential of advances in cysteine protease inhibitor design, Daniel F Veber and Scott K Thompson, Current Opinion in Drug Discovery & Development, 2000, 3, 362-369. Specific examples of unnatural amino acids used in the exploration of receptor binding are shown in WO 9740065 and WO99231 09. A specific example of a therapeutic peptidomimetic employing a non-natural, branched amino acid is found in our copending application PCT/GB00/01894 with priority from GB 9911417.

The contents of the references in the above paragraph are specifically incorporated by reference.

The application of the invention can be illustrated by way of example only with reference to the following representative compounds 3-7 of the invention and their precursors 1 and 2. The illustrated Fmoc derivatives are readily amenable to automated peptide synthesis.

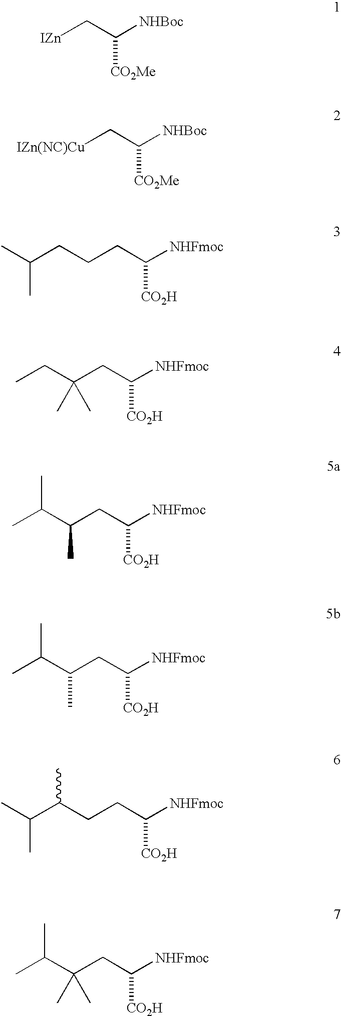

The invention envisages the copper-promoted reaction of zinc reagent 1 with highly substituted allylic electrophiles. In our original work, ² we had employed the stoichiometric transmetallation of the zinc reagent 1 to the zinc/copper reagent 2 using CuCN.2LiCl, prior to addition of the electrophile. While this process is reliable, the need to exercise appropriate precautions during the reaction due to the toxicity of cyanide, and especially during the work-up, is a significant drawback. This prompted us to explore the use of catalytic amounts of copper, most specifically CuBr.DMS, which has recently been reported to catalyse the reaction between β-amino zinc reagents and allenic halides.⁷,⁸ In addition, we were concerned that the electrophiles that we proposed to use, 8-10, might be susceptible to copper-catalysed isomerisation in the presence of halide ion, which in turn would lead to mixtures of products provided the usual S_{N}2′ pathway was followed in the substitution. The use of catalytic amounts of copper is now shown to minimise this problem.

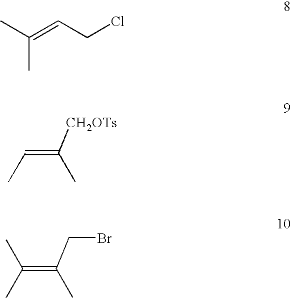

Reaction of the zinc/copper reagent, prepared under our previously described conditions¹⁻⁴, with 3,3-dimethylallyl chloride gave a mixture of the constitutional isomers 11 and 12, in a 58:42 ratio (93%). When the zinc reagent 1 was treated with 3,3-dimethylallyl chloride in the presence of a catalytic amount of CuBr.DMS, the two isomers 11 and 12 were isolated in excellent overall yield (90%), and in a ratio of 55:45. These results suggest that while the work-up can be much simplified by the use of catalytic amounts of copper, the regiochemical outcome of the reaction is not altered. Unfortunately, it did not prove possible to separate 11 and 12, so we took advantage of the higher reactivity of trisubstituted alkenes, compared with terminal alkenes, towards m-CPBA.⁹ Thus, treatment of the mixture of 11 20 and 12 with m-CPBA resulted in selective epoxidation of 11 to give 13 (as a mixture of diastereoisomers), leaving 12 untouched. The separation of alkene 12 from epoxide 13 proved straightforward, and epoxide 13 was converted back into the terminal alkene 11 by treatment with the reagent derived from WCl₆/BuLi (Scheme 1).¹⁰,¹¹

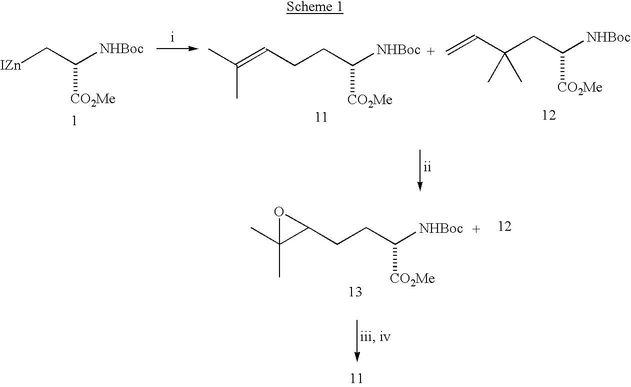

Reagents and conditions: i, CuBr.DMS, (CH₃)₂C═CHCH₂Cl; ii, m-CPBA, CHCl₃, room temp., 2 h; iii, separation; iv, WCl₆/BuLi, −78° C., then 0-5° C., 30 min, room temp., 1 h.

Separate hydrogenation of compounds 11 and 12 proceeded smoothly to give the saturated analogues 14 and 15. These two compounds were fully characterised, and then converted into the Fmoc-protected amino acids 3 and 4 by a series of standard protecting group manipulations (Scheme 2).

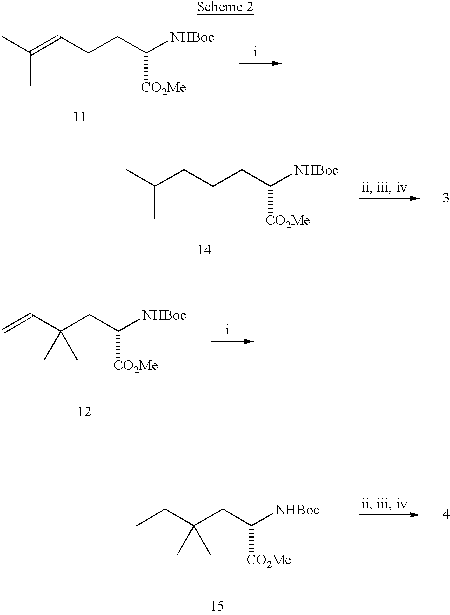

Reagents and conditions: i, H₂, Pd/C, EtOH, room temp.; ii, LiOH, THF/H₂O, 1:1, room, temp.; iii, HCl (4 M), dioxane; iv, FmocCl, Na₂CO₃, H₂O, dioxane, room temp.

In order to prepare the two diastereoisomers 5a and 5b, it was necessary to treat the zinc reagent 1 with the tosylate 9, which was prepared in two steps from tiglic acid.¹²,¹³ Tosylate 9, as reported in the literature,¹³ is very unstable, and it is necessary to store the compound in solution. Nevertheless, the CuBr.DMS catalysed reaction gave the separable diastereoisomers 16a (32%) and 16b (19%) in moderate combined yield. The relative stereochemistry of the racemic N-acetyl analogues of 16a and 16b, prepared by a Lewis acid catalysed ene reaction between methyl 2-acetamidoacrylate and 2-methyl-2-butene, has been tentively assigned by analogy with the outcome of a related reaction.¹⁴ By comparison of the published ¹³C NMR data of these N-acetyl analogues¹⁴ with that for 16a and 16b (specifically the chemical shift of the terminal methylene carbon), we have tentatively assigned the stereochemistry of 16a as anti, and 16b as syn. Compounds 16a and 16b were then separately converted in an analogous series of steps to those already described, via the characterised saturated analogues 17a and 17b, into the target Fmoc-protected acids 5a and 5b (Scheme 3).

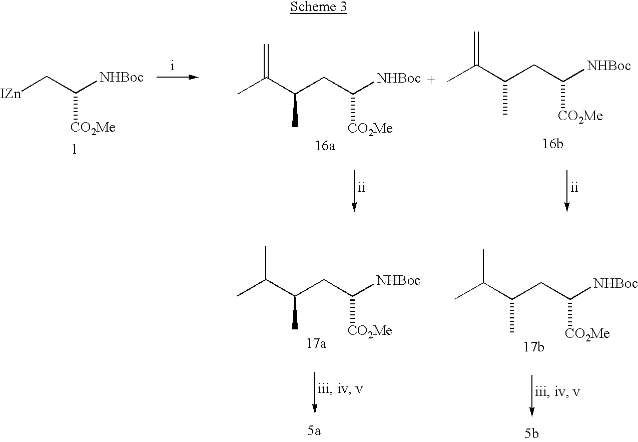

Reagents and conditions: i, CuBr.DMS, E-CH₃CH═C(CH₃)CH₂OTs; ii, H₂, Pd/C, EtOH, room temp.; iii, LiOH, THF/H₂O, 1:1, room, temp.; iv, HCl (4 M), dioxane; v, FmocCl, Na₂CO₃, H₂O, dioxane, room temp.

With the aim of preparing the homologues of compounds 5a and 5b, the copper-catalysed reaction of the zinc reagent 1 with the bromide 10, prepared by HBr addition to 2,3-dimethylbutadiene,¹⁵ was investigated. The two constitutional isomers 18 (29%) and 19 (30%) were isolated, and these could be separated by flash chromatography. This reaction was carried out on a 30 mmol scale, and demonstrates the capability of this method to prepare gram amounts of material. The unsaturated amino acids 18 and 19 were then converted via the saturated analogues 20 (isolated as an inseparable mixture of diastereoisomers) and 21, and the derived Boc-protected amino acids 22 and 23 into the targets 6 (also isolated as an inseparable mixture of diastereoisomers) and 7, respectively.

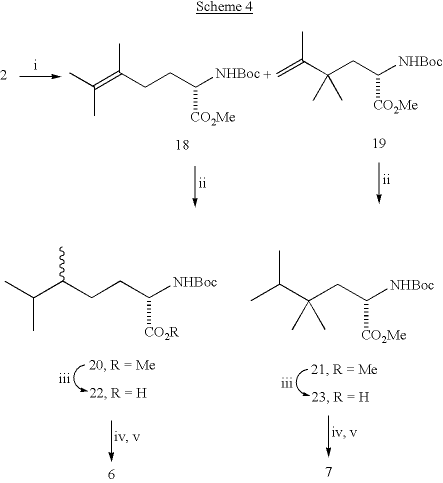

Reagents and conditions: i, CuBr.DMS, (CH₃)₂C═C(CH₃)CH₂Br; ii, H₂, Pd/C, EtOH, room temp.; iii, LiOH, THF/H₂O, 1:1, room, temp.; iv, HCl (4 M), dioxane; v, FmocCl, Na₂CO₃, H₂O, dioxane, room temp

It is apparent form the representative compounds and syntheses above that the normal course of substitution reactions of allylic electrophiles with zinc/copper reagents, in which the products from the S_{N}2′ pathway predominate, is no longer followed when highly substituted electrophiles are used. Electrophiles in which the S_{N}2′ pathway would require attack at a fully substitued position, as is the case for 8 and 10, tend to give significant amounts of the products formally derived by the S_{N}2 pathway. At this stage, we cannot rule out the possibility that the products formally derived by the S_{N}2 pathway actually arise by an initial isomerisation of the electrophile (which is known to be promoted by copper salts, even if these are present only in sub-stoichiometric amounts), rather than an S_{N}2.

From a preparative point of view, we have shown how the copper-catalysed reaction of the serine-derived zinc reagent 1 with substituted allylic electrophiles can be used to good effect in the preparation of a series of amino acids with branched hydrophobic side-chains. Although conventional isomers are formed, these can be separated by concentional techniques.

Accordingly a further aspect of the invention envisages a method of synthesising a compound of the formula I

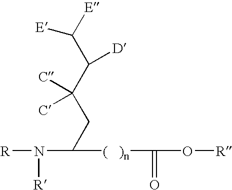

wherein

R are independently H or an amine protecting group;

R′ is C₁-C₆ alkyl, C₂-C₆ alkenyl, ArC₀-C₆ alkyl or HetC₀-C₆ alkyl,

R″ is H or a carboxy protecting group;

( ) is a methylene group;

n is 0,1 or 2;

C′, C″, D′, E′ and E′ are hydrogen (H) or a group selected from C₁-C₆ alkyl, C₂-C₆ alkenyl, ArC₀-C₆ alkyl or HetC₀-C₆ alkyl, (“Alk”) in the following permutations:

| C′ | C″ | D′ | E′ | E″ |
|---|---|---|---|---|
| H | H | H | Alk | Alk |
| H | H | Alk | Alk | Alk |
| H | Alk | Alk | H | H |
| Alk | Alk | H | H | H |
| Alk | Alk | Alk | H | H |
| Alk | H | H | H | H |

comprising the steps of reacting a zinc reagent of the formula:

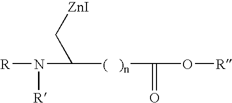

wherein R is an amine protecting group, R′ is H, C₁-C₆ alkyl, C₂-C₆ alkenyl, ArC₀-C₆ alkyl or HetC₀-C₆ alkyl, and R′ is a carboxy protecting group, with an allylic electrophile; separation of isomers, hydrogenation of the double bond and deprotection as necessary.

The separation may comprises the selective epoxidation of a compound of the formula:

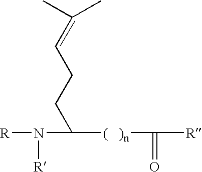

where R, R′, R″, ( ) and n are as defined above.

Although the invention has been illustrated above and in the accompanying Examples by reference to compounds wherein Alk is methyl, it will be apparent that the corresponding branched allyls corresponding to 8, 9 and 10, but with the appropriate permutations of Alk variables, such as C₂-C₆ alkyl, C₂-C₆ alkenyl, ArC₀-C₆allyl or HetC₀-C₆alkyl will be amenable to corresponding synthesis. These branched allyls are readily obtained commercially or by facile modifications of commerically available starting products. Functionalities optionally present as substituents on the Alk moiety will generally be protected with conventional protecting groups prior to the manipulations envisaged in the method of the invention.

Although the illustrative embodiments employ Fmoc as the ultimate amino protecting group as the chemistry of peptide and peptidomimetic synthesis is well established, it will be apparent that a wide range of alternative protecting groups are available, including those specified below. The compounds of the invention may alternatively be carboxy-protected with conventional protecting groups as outlined below to facilitate reactions at the alpha amine.

Although the illustrative embodiments employ an L-serine derived organozinc reagent to produce alpha L-amino acids, it will be apparent that employment of the readily available corresponding acids L-3-amino-4-hydroxybutyric acid and L-4-amino-5-hydroxy-pentanoic acid will produce beta and gamma amino acids with the desired stereochemistry at the alpha carbon. Similarly use of the corresponding D acids will provide pure or at least enriched D stereochemistry at the alpha carbon.

It will be apparent that unsaturated compounds 11, 12, 16a, 16b, 18 and 19 in addition to their use as intermediates will also be useful as unnatural amino acids in the same way as the other compounds of the invention.

C₀ or C₁-C₆alkyl as applied herein includes straight and branched chain aliphatic carbon chains such as methyl, ethyl, n-propyl, isopropyl, n-butyl, isobutyl, t-butyl, pentyl, isopentyl, hexyl, heptyl, or cycloalkyls, optionally bonded through C₁-C₃ alkyl. Additionally, any C₁-7-alkyl may optionally be substituted by one or two halogens and/or a heteroatom S, O, NH. If the heteroatom is located at a chain terminus then it is appropriately substituted with one or 2 hydrogen atoms.

‘C1-3-alkyl’ as applied herein includes methyl, ethyl, propyl, isopropyl, cyclopropyl, any of which may be optionally substituted as described in the paragraph above.

‘Amine’ includes NH2, NHC1-3-alkyl or N(C1-3-alkyl)2.

‘Halogen’ as applied herein is meant to include F, Cl, Br, I, particularly chloro and preferably fluoro.

‘ArC₀-C₆-alkyl’ as applied herein includes a phenyl or napthyl attached through a C1-6-alkyl (defined above). Optionally, the aromatic ring Ar may be substituted with halogen, C1-3-alkyl, OH, OC1-3-alkyl, SH, SC1-3-alkyl, amine and the like, it being understood that such optional functionalities will generally be protected or masked with conventional protecting groups prior to the manipulations envisaged in the method of the invention.

HetC₀-C₆ alkyl as applied herein includes aromatic and non-aromatic moieties such as piperidinyl, piperazinyl, pyrrolidinyl, azepinyl, thienyl, pyrrolyl, pyrrolidinyl, pyrazolyl, pyrazolidnyl, imidazolyl, pyridyl, pyrazinyl, oxazolinyl, oxazolyl, isooxazolyl, morpholinyl, thiazolinyl, isothiazolyl, thiazolyl, quinuclidinyl, indolyl, quinolyl, isoquinolyl, benzimidazolyl, benzothienyl, benzopyranyl, benzoxazolyl, benzofuranyl, furyl, pyranyl, tetrahydrofuryl, tetrahydropyranyl, theinyl, oxadiazolyl, benzothiazolyl, benzoisathiazolyl, benzoxazolyl, pyrimidinyl, cinolyl, quinazolyl, quinoxalinyl, tetrazolyl, triazolyl and the like, which are linked through a C₀-C₆ alkyl as defined in the paragraph immediately above.

The term “N-protecting group” or “N-protected” and the like as used herein refers to those groups intended to protect the N-terminus of an amino acid or peptide or to protect an amino group against undesirable reactions during synthetic procedures. Commonly used N-protecting groups are disclosed in Greene, “Protective Groups in Organic Synthesis” (John Wiley & Sons, New York, 1981), which is hereby incorporated by reference. N-protecting groups include acyl groups such as formyl, acetyl, propionyl, pivaloyl, t-butylacetyl, 2-chloroacetyl, 2-bromoacetyl, trifluoracetyl, trichloroacetyl, phthalyl, o-nitrophenoxyacetyl, c-chlorobutyryl, benzoyl, 4-chlorobenzoyl, 4-bromobenzoyl, 4-nitrobenzoyl, and the like; sulfonyl groups such as benzenesulfonyl, p-toluenesulfonyl, and the like, carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butoxycarbonyl, diisopropylmethoxycarbonyl, isopropyloxycarbonyl, ethoxycarbonyl, methoxycarbonyl, allyloxycarbonyl, 2,2,2-trichloroethoxycarbonyl, phenoxycarbonyl, 4-nitrophenoxycarbonyl, fluorenyl-9-methoxycarbonyl, (Fmoc ), cyclopentyloxycarbonyl, adamantyloxycarbonyl, cyclohexyloxycarbonyl, phenylthiocarbonyl, and the like; alkyl gropus such as benzyl, triphenylmethyl, benzyloxymethyl and the like; and silyl groups such as trimethylsilyl and the like. Favoured N-protecting groups include formyl, acetyl, allyl, Fmoc, benzoyl, pivaloyl, t-butylacetyl, phenylsulfonyl, benzyl, t-butoxycarbonyl (BOC) and benzyloxycarbonyl (Cbz).

Hydroxy and/or carboxy protecting groups are also extensively reviewed in Greene ibid and include ethers such as methyl, substituted methyl ethers such as methoxymethyl, methylthiomethyl, benzyloxymethyl, t-butoxymethyl, 2-methoxyethoxymethyl and the like, silyl ethers such as trimethylsilyl (TMS), t-butyldimethylsilyl (TBDMS) tribenzylsilyl, triphenylsilyl, t-butyldiphenylsilyl triisopropyl silyl and the like, substituted ethyl ethers such as 1-ethoxymethyl, 1-methyl-1-methoxyethyl, t-butyl, allyl, benzyl, p-methoxybenzyl, dipehenylmethyl, triphenylmethyl and the like, aralkyl groups such as trityl, and pixyl (9-hydroxy-9-phenylxanthene derivatives, especially the chloride). Ester hydroxy protecting groups include esters such as formate, benzylformate, chloroacetate, methoxyacetate, phenoxyacetate, pivaloate, adamantoate, mesitoate, benzoate and the like. Carbonate hydroxy protecting groups include methyl vinyl, allyl, cinnamyl, benzyl and the like.

### DETAILED DESCRIPTION

### EXAMPLE 1

a) General Procedures

Dry DMF was distilled from calcium hydride and stored over 4 Å molecular sieves. Dry dichloromethane was distilled from calcium hydride. Dry THF was distilled from potassium benzophenone ketyl. Petroleum ether refers to the fraction with a boiling point between 40-60° C. Specific rotations were measured at 20° C., unless otherwise stated. IR spectra (nmax) were recorded on a Nicolet 20PCIR spectrometer at University of Newcastle as thin films. Mass Spectra (m/z) (ESP⁺) were obtained using a Fisons/VG analytical system at Medivir UK, Cambridge or measured on a Micromass Autospec M spectrometer in E.I. mode at the University of Newcastle. HRMS mass spectra (m/z) (ESP⁺) were recorded using a Q-TOF Micromass spectrometer by University of Cambridge Spectrometry Service or a Micromass Autospec M spectrometer in E.I. mode at the University of Newcastle. Nuclear Magnetic Resonance (NMR) spectra were recorded at the field strength in the solvents indicated, using standard pulse sequences on a DRX-500 machine by University of Cambridge NMR Department or on a Bruker AC 200 (200 MHz) or JEOL LA 500 (500 MHz) instrument at University of Newcastle. Chemical shifts are expressed in parts per million (d) and are referenced to residual signals of the solvent. Coupling constants (J) are expressed in Hz. Elemental analyses were carried out either by University of Cambridge Microanalysis Service or by University of Newcastle Microanalysis Service. Unless otherwise specified, all solvents and reagents were obtained from commercial suppliers and used without further purification. HPLC samples were run on a Vydac Phenomenex Jupiter C₄ (5 m)250×4.6 mm analytical column using an automated Gilson 215/233XL. A gradient of 10-90% B in A, 2-30 min, 1.5 cm³/min, where solvent A=0.1 % aq TFA and solvent B=acetonitrile/10% A, with UV detection at 215 nm Thin Layer Chromatography (TLC) was performed on precoated plates (Merck aluminium sheets silica 60 F254, Art. no. 5554). Visualisation of compounds was achieved by illumination under ultraviolet light (254 nm) or using an appropriate staining reagent. Flash Column Chromatography was performed on Silica Gel 60 (Merck 9385).

b) General Zinc Coupling Reactions:

b i) Zinc Activation:

Zinc dust (150 mg, 2.29 mmol, 3.0 eq, Aldrich) was weighed into a 25 cm³ round bottom flask with a side arm and fitted with a three way tap. The zinc powder was heated with a heat gun under vacuum and the flask was flushed with nitrogen and evacuated and flushed a further three times. With the flask filled with nitrogen, dry DMF (1 cm³) was added. Trimethylsilylchloride (0.029 cm³, 0.23 mmol, 0.3 eq) was added and the zinc slurry was vigorously stirred for a further 30min.

b ii) Zinc Insertion:

N-(tert-Butoxycarbonyl)-3-iodo-L-alanine methyl ester² (247 mg, 0.75 mmol, 1.0 eq) dissolved in dry DMF (0.5 cm³) was added dropwise, via cannula, to the activated zinc slurry at 0° C. prepared as described above. The reaction mixture was then allowed to warm up to room temperature and stirred for 1 h to give the organozinc reagent.

b iii) CuBr.SMe₂ Preparation:

Whilst the zinc insertion reaction was in progress, CuBr.SMe₂ (21 mg, 0.10 mmol, 0.13 eq) was weighed into a 25 cm³ round bottom flask fitted with a three way tap and dried gently with a heat gun under vacuum until CuBr.SMe₂ changed appearance from a brown powder to a light green powder. Dry DMF (0.5 cm³) was then added followed by addition of the electrophile (1-chloro-2-methylbut-2-ene, toluene4-sulfonic acid-(E)-2-methyl-but-2-enyl ester or 1-bromo-2,3-dimethylbut-2-ene) (1.00 mmol, 1.3 eq). The reaction mixture was then cooled to −15° C.

b iv) Coupling Reaction:

Stirring of the organozinc reagent solution was stopped to allow the zinc powder to settle and the supernatant was carefully removed via syringe (care taken to avoid transferring too much zinc powder) and added dropwise to the solution of electrophile and copper catalyst. The cooling bath was removed and the solution was stirred at room temperature overnight. Ethyl acetate (20 cm³) was added and stirring was continued for a further 15 min. The reaction mixture was transferred to a separating funnel and a further aliquot of EtOAc (30 cm³) was added. The organic phase was washed successively with 1M Na₂S₂O₃ (20 cm³), water (2×20 cm³), brine (40 cm³), dried (Na₂SO₄ or MgSO₄) and filtered. The solvent was removed in vacuo and the crude product purified by flash chromatography on silica gel as described.

c) Hydrogenation of Alkene:

The alkene (1.00 mmol) was dissolved in ethanol (10 cm³), 10% palladium on carbon (80 mg) added and hydrogen introduced. Once the reaction had been judged to have reached completion (tlc, hplc or MS), the hydrogen was removed, the reaction filtered through Celite and the catalyst washed with ethanol (30 cm³). The combined organic filtrate was concentrated in vacuo and the alkane used directly in the subsequent reaction or purified by flash chromatography on silica gel as described.

d) Saponification of Methyl Ester:

The methyl ester (1.00 mmol) was dissolved in THF (6 cm³) and whilst stirring, a solution of LiOH (1.20 mmol, 1.2 eq) in water (6 cm³) was added dropwise. Once the reaction was judged to have reached completion (tlc, hplc or MS), the THF was removed in vacuo and diethyl ether (10 cm³) added to the residue. The reaction mixture was acidified with 1.0 M HCl until pH 3. The organic phase was then removed and the aqueous layer extracted with diethyl ether (2×10 cm³). The combined organic extracts were dried over magnesium sulphate, filtered and the solvent removed in vacuo to give the carboxylic acid used directly in the subsequent reaction or purified by flash chromatography on silica gel as described.

e) Removal of N-Boc Protecting Group:

The N-Boc protected material (1.00 mmol) was cooled to 0° C. and 4 M HCl in dioxane (5 cm³) added dropwise and when the reaction was judged to have reached completion (tlc, hplc or MS), the solvents were removed in vacuo to yield the amine hydrochloride used directly in the subsequent reaction.

f) Fmoc Protection of Amine:

The amine (1.00 mmol) in 1,4-dioxane (2 cm³) was cooled to 0° C. and 10% sodium carbonate (2.20 mmol, 2.2 eq, 4 cm³) added. The biphasic reaction mixture was stirred vigorously and Fmoc-Cl (1.10 mmol, 1.1 eq) in dioxane (2 cm³) was added over 1 h. Once the reaction was judged to have reached completion (tlc, hplc or MS), diethyl ether (10 cm³) was added and the reaction mixture acidified to pH 3 with 1 M HCl. The organic phase was removed and the aqueous layer extracted with diethyl ether (2×10 cm³). The combined organic extracts were dried over sodium sulphate, filtered, the solvent removed in vacuo and the residue purified by flash chromatography using silica gel.

### EXAMPLE 2

2S-2-(9H-Fluoren-9-ylmethoxycarbonylamino)4,4-dimethyl-hexanoic acid 4.

a) 2S-2-tert-Butoxycarbonylamino-4,4-dimethyl-hex-5-enoic acid methyl ester 12;

2S-2-tert-butoxycarbonylamino-4-(2S-3,3-dimethyl-oxiranyl)-butyric acid methyl ester 13a; and

2S-2-tert-butoxycarbonylamino-4-(2R-3,3-dimethyl-oxiranyl)-butyric acid methyl ester 13b.

Following the general procedure for zinc coupling reactions, 1-chloro-3-methylbut-2-ene (0.110 cm³, 0.98 mmol) was coupled to N-(tert-butoxycarbonyl)-3-iodo-L-alanine methyl ester (247 mg, 0.75 mmol) in the presence of CuBr.SMe₂ (21 mg, 0.10 mmol) to give a residue which was purified by flash column chromatography over silica gel eluting with EtOAc/heptane (1:9, v/v). Fractions were pooled and reduced in vacuo to give on the basis of ¹H NMR spectroscopy a mixture of regioisomers (183 mg, 90%) (45:55 formal S_{N}2′ vs S_{N}2), inseparable by column chromatography, as a colourless oil.

To a mixture of isomers 11 and 12 (190 mg, 0.70 mmol) in chloroform (3 cm³) was added dropwise over 5 min, 3-chloroperbenzoic acid (164 mg, 85% pure, 0.81 mmol, 1.15 eq) in chloroform (2 cm³). The reaction mixture was stirred at, room temperature for a further 2 h. The reaction mixture was then washed successively with 1 M Na₂S₂O₅ (5 cm³), saturated sodium bicarbonate solution (5 cm³) and brine (10 cm³). The organic phase was dried over sodium sulfate, filtered, the solvent removed in vacuo and the residue was purified by flash chromatography over silica gel eluting with EtOAc/heptane (1:9, v/v). Three products were obtained; 2S-2-tert-butoxycarbonylamino-4,4-dimethyl-hex-5-enoic acid methyl ester 12 was eluted first and further elution afforded an inseparable mixture of 2S-2-tert-butoxycarbonylamino-4-(2S-3,3-dimethyl-oxiranyl)-butyric acid methyl ester 13a and 2S-2-tert-butoxycarbonylamino-4-(2R-3,3-dimethyl-oxiranyl)-butyric acid methyl ester 13b. Fractions containing the initial component were pooled and reduced in vacuo to give 2S-2-tert-butoxycarbonylamino-4,4-dimethyl-hex-5-enoic acid methyl ester 12 (93 mg, 49%) as a colourless oil.

Analytical HPLC Rt=21.45 min (95%); [a]_{D18}+18.7 (c 0.32 in CH₂Cl₂);

ₙₘₐₓ(film)/cm⁻¹ 3369 (s), 3084 (m), 2965 (s), 1748 (s), 1715 (s), 1517 (s), 1167 (s), 1007 (s) and 914 (s); δ_{H}(500 MHz; CDCl₃) 1.06 (6H, s, CH₂═CHC(CH₃)₂), 1.42 (9H, s, C(CH₃)₃) 1.55 (1H, dd, J 14 and 9, NHCHCH_{2A}), 1.82 (1H, dd, J 14 and 4, NHCHCH_{2B}), 3.69 (3H, S, CO₂CH₃), 4.30 (1H, br m, NHCHCO₂CH₃), 4.83 (1H, br d, J 7, NH), 4.97 (2H, m, CH₂═CH) and 5.78 (1H, dd, Jₜᵣₐₙₛ 17.5 and J_{cis} 11, CH₂═CH); δ_{C}(125 MHz; CDCl₃) 26.93 (CH₂═CHC(CH₃)₂), 28.34 (C(CH₃)₃), 36.33 (CH₂═CHC(CH₃)₂), 45.06 (NHCHCH₂), 51.25 (NHCHCO₂CH₃), 52.15 (CO₂CH₃), 79.77 (C(CH₃)₃), 111.39 (CH₂═CH), 146.87 (CH₂═CH), 154.97 (OC(O)NH) and 174.04 (NHCHCO₂CH₃); hrms 215.1152 (M⁺-C₄H₈.C₁₀H₁₇NO₄ requires 215.1158 (d 2.8 ppm)); m/z (Electrospray-MS) 272 (40%) and 216 (100%).

Pooling together the lower eluting component gave a mixture of 2S-2-tert-butoxycarbonylamino-4-(2S-3,3dimethyl-oxiranyl)butyric acid methyl ester 13a and 2S-2-tert-butoxycarbonylamino-4-(2R-3,3-dimethyl-oxiranyl)-butyric acid methyl ester 13b (55 mg, 27%) as a colourless oil. (¹H NMR spectroscopy showed a mixture of diastereoisomers had been obtained in a 3.5:1 ratio. No attempt was made to establish which isomer was formed preferentially).

[α]_{D}²³+12.0 (c 1.02 in CH₂Cl₂); ₙₘₐₓ(film)/cm⁻¹ 2976 (br), 2931 (s), 1747 (s), 1716 (s), 1391 (s) and 1367 (s); δ_{H} (500 MHz; CDCl₃) 1.26 (3H, s, (CH₃)_{2A}), 1.31 (3H, s, (CH₃)_{2B}), 1.44 (9H, s, C(CH₃)₃),), 1.52 (1H, m, NHCHCH₂CH_{2A}), 1.61 (1H, m, NHCHCH₂CH_{2B}), 1.80 (1H, m, NHCHCH_{2A}CH₂), 2.01 (1H, m, NHCHCH_{2B}CH₂), 2.69 (1H, dd, J 7 and 5.5, NHCH(CH₂)₂CH), 3.75 (3H, s, CO₂CH₃), 4.35 (1H, br m, NHCHCO₂CH₃) and 5.20 (1H, br d, J 8, NH); δ_{C} (125 MHz; CDCl₃) 18.61 and 18.62 ((CH₃)_{2A}), 24.77 and 24.79 (NHCHCH₂CH₂), 24.81 and 25.08 ((CH₃)_{2B}), 28.30 (C(CH₃)₃), 29.51 and 29.61 (NHCHCH₂CH₂), 52.30 and 52.36 ((CH₃)₂CCH), 53.08 and 53.27 ((NHCHCH₂), 58.55 (CO₂CH₃), 63.36 and 63.48 ((CH₃)₂C), 79.87 (C(CH₃)₃), 155.38 (OC(O)NH) and 173.01 (NHCHCO₂CH₃); hrms 288.1823 (MH⁺. C₁₄H₂₆NO₅ requires 288.1811 (d 4.2 ppm)); m/z (Electrospray-MS) 288 (91 %) and 232 (100%).

b) ²S-²-tert-Butoxycarbonylamino-4,4-dimethyl-hexanoic acid methyl ester 15:

Following the general procedure for alkene hydrogenation, 2S-2-tert-butoxycarbonylamino-4,4-dimethyl-hex-5-enoic acid methyl ester 12 (93 mg, 0.34 mmol) yielded on purification by flash column chromatography over silica gel, eluting with EtOAc/heptane (1:5, v/v), 2S-2-tert-butoxycarbonylamino-4,4-dimethyl-hexanoic acid methyl ester 15 (90 mg, 96%) as a colourless oil.

Analytical HPLC Rt=22.55 min (100%); _{[a]D18}−6.1 (c 0.99 in CH₂Cl₂); δ_{H} (500 MHz; CDCl₃) 0.81 (3H, t, J 7.5, (CH₃CH₂), 0.89 (3H, s, CH₃CH₂C(CH₃)_{2A}), 0.90 (3H, s, CH₃CH₂C(CH₃)_{2B}), 1.29 (2H, dq, J 7.5 and 1, CH₃CH₂), 1.38 (1H, dd, J 14.5 and 9, NHCHCH_{2A}), 1.42 (9H, s, C(CH₃)₃), 1.69 (1H, dd, J 14.5 and 3.5, NHCHCH_{2B}), 3.71 (3H, s, CO₂CH₃), 4.31 (1H, br m, NHCHCO₂CH₃) and 4.78 (1H, br d, J 8.5, NH); δ_{C} (125 MHz; CDCl₃) 8.66 (CH₃CH₂), 26.61 (CH₃CH₂C(CH₃)₂), 28.28 (C(CH₃)₃), 33.06 (CH₃CH₂C)(CH₃)₂), 34.40 (CH₃CH₂ ), 43.97 (NHCHCH₂), 50.84 ((NHCHCH₂), 52.13 (CO₂CH₃), 79.79 (C(CH₃)₃), 155.08 (OC(O)NH) and 174.46 (NHCHCO₂CH₃); hrms 296.1827 (MNa.C₁₄H₂₇NO₄Na requires 296.1838 (_{d} 3.7 ppm)); m/z (Electrospray-MS) 274 (69%) and 218 (100%).

c) 2S-2-ter-Butoxycarbonylamino-4,4-dimethyl-hexanoic acid:

Following the general procedure for methyl ester saponification, 2S-2-tert-butoxycarbonylamino-4,4-dimethyl-hexanoic acid methyl ester 15 (90 mg, 0.33 mmol) gave 2S-2-tert-butoxycarbonylamino-4,4-dimethyl-hexanoic-acid (79 mg, 93%) as crystals and used directly in the subsequent reaction.

Analytical HPLC Rt=20.90 min (100%); m/z (Electrospray-MS) 260 (33%) and 204 (100%).

d) 2S-2-Amino-4,4-dimethyl-hexanoic acid hydrochloride salt:

Following the general procedure of N-Boc removal using 4 M HCl in dioxane, 2S-2-tert-butoxycarbonylamino-4,4-dimethyl-hexanoic acid (79 mg, 0.31 mmol) gave 2S-2-amino-4,4-dimethyl-hexanoic acid hydrochloride salt (60 mg, 100%) as a solid, and used directly in the subsequent reaction; m/z (Electrospray-MS) 160 (100%).

e) 2S-2-(9H-Fluoren-9-ylmethoxycarbonylamino)-4,4-dimethyl-hexanoic acid 4:

Following the general procedure for Fmoc protection of an amine, 2S-2-amino-4,4-dimethyl-hexanoic acid hydrochloride salt (60 mg, 0.31 mmol) gave on purification by flash chromatography over silica gel, eluting with CHCl₃/CH₃OH (100:0 to 96:4, v/v), 2S-2-(9H-fluoren-9-ylmethoxycarbonylamino)-4,4dimethyl-hexanoic acid 4 (63 mg, 54%) as an amorphous solid, mp 64-65° C.

Analytical HPLC Rt=23.63 min (100%); [α]_{D}¹⁸−17.4 (c 1.01 in CH₂Cl₂); δ_{H} (500 MHz; CDCl₃) 0.82 (3H, t, J 7.5, CH₃CH₂), 0.91 (3H, s, C H₃CH₂C(CH₃)_{2A}), 0.92 (3H, s, CH₃CH₂C(CH₃)_{2B}), 1.29 (2H, br q, J 7.5, CH₃CH₂), 1.46 (1H, dd, J 14.5 and 9.5, NHCHCH_{2A}), 1.83 (1H, dd, J 14.5 and 2, NHCHCH_{2B}), 4.20 (1H, t, J 7, H-9′), 4.40 (3H, br m, NHCHCO₂H and CH₂O), 5.07 (1H, br d, J 7.5, NH), 7.28 (2H, m, H-2′ and H-7′), 7.37 (2H, m, H-3′ and H-6′), 7.56 (2H, m, H-1′ and H-8′) and 7.74 (2H, d, J 7.5, H4′ and H-5′); dC (125 MHz; CDCl₃) 8.23 (CH₃CH₂), 26.62 (CH₃CH₂C(CH₃)₂), 33.20 (CH₃CH₂C(CH₃)₂), 34.37 (CH₃CH₂), 43.40 (NHCHCH₂), 47.14 (CH-9′), 51.30 (NHCHCO₂H), 67.01 (CH₂O), 119.92 (CH4′ and CH-5′), 124.99 (CH-1′ and CH-8′), 127.01 (CH-2′ and CH-7′), 127.65 (CH-3′ and CH-6′), 141.27 (C4a′ and C-5a′), 143.70 (C-1a′ and C-8a′), 155.90 (OC(O)NH) and 177.07 (NHCHCO₂H); hrms 404.1839 (MNa.C₂₃H₂₇NO₄Na requires 404.1838 (d 0.2 ppm)); m/z (Electrospray-MS) 382 (100%).

### EXAMPLE 3

2S-2-(9H-Fluoren-9-ylmethoxycarbonylamino)-6-methyl-heptanoic acid 3

a) 2S-2-tert-Butyloxycarbonylamino-6-methyl-hept-5-enoic methyl ester 11:

Hexachlorotungsten (106 mg, 0.30 mmol, 1.4 eq) was weighted out into a Schlenk tube under nitrogen and dry THF (0.5 cm³) was added. A solution of nBuLi (0.216 cm³, 2.5 M, 0.60 mmol, 2.8 eq) was added dropwise to the tungsten solution at −78° C. and the solution was then left to warm up slowly to room temperature to give a clear brown solution. It was then recooled to −78° C. and treated with a solution of 2S-2-tert-butoxycarbonylamino4-(2S-3,3-dimethyl-oxiranyl)-butyric acid methyl ester 13a and 2S-2-tert-butoxycarbonylamino-4-(2R-3,3-dimethyl-oxiranyl)-butyric acid methyl ester 13b (55 mg, 0.19 mmol) in THF (0.2 cm³). The reaction mixture was stirred at 0-5° C. for 30 min and then at room temperature for 1 h to give a clear green solution. The reaction mixture was poured into a 1:1 solution of 1.5 M sodium tartrate and 2 M sodium hydroxide (5 cm³). The organic layer was removed and dried over magnesium sulphate, filtered and the solvent removed in vacuo to give a crude oil. The residue was purified by flash chromatography over silica gel eluting with EtOAc/heptane (1:5, v/v) to give 2S-2-tert-butyloxycarbonylamino-6-methyl-hept-5-enoic methyl ester 11 (25 mg, 48%) as a colourless oil.

Analytical HPLC Rt=21.32 min (100%); ₙₘₐₓ(film)/cm⁻¹ 3364 (m), 2977 (m), 1744 (s), 1715 (s), 1516 (s) and 1167 (s); [α]_{D}^{18+11.9} (c 1.01 in CH₂Cl₂); δ_{H} (500 MHz; CDCl₃) 1.43 (9H, s, C(CH₃)₃) 1.59 (3H, s, (CH₃)_{2A}C═CH), 1.64 (1H, m, NHCHCH₂CH_{2A}), 1.68 (3H, s, (CH₃)_{2B}C═CH), 1.82 (1H, m, NHCHCH₂CH_{2B}), 2.01 (1H, dd, J 14.5 and 7.5, NHCHCH_{2A}), 2.06 (1H, dd, J 14.5 and 6.5, NHCHCH_{2B}), 3.73 (3H, s, CO₂CH₃), 4.30 (1H, br m, NHCHCO₂CH₃), 4.99 (1H, br d, J 7.0, NH) and 5.07 (1H, br t, J 7.0, (CH₃)₂C═CH); δ_{C} (125 MHz; CDCl₃) 17.65 ((CH₃)_{2A}C═CH), 23.89 (NHCHCH₂CH₂), 25.71 ((CH₃)_{2B}C═CH), 28.33 (C(CH₃)₃), 32.67 (NHCHCH₂), 52.19 (CO₂CH₃), 53.15 (NHCHCO₂CH₃), 79.53 (C(CH₃)₃), 122.68 ((CH₃)₂C═CH), 132.89 ((CH₃)₂C═CH), 155.21 (OC(O)NH) and 173.24 (NHCHCO₂CH₃); hrms 294.1687 (MNa. C₁₄H₂₅NO₄Na requires 294.1681 (d 1.8 ppm)); m/z (Electrospray-MS) 272 (100%).

b) 2S-2-tert-Butoxycarbonylamino-6-methyl-heptanoic acid methyl ester 14:

Following the general procedure for alkene hydrogenation, 2S-2-tert-butyloxycarbonylamino-6-methyl-hept-5-enoic methyl ester 11 (48 mg, 0.18 mmol) yielded on purification by flash column chromatography over silica gel, eluting with EtOAc/heptane (1:10, v/v), 2S-tert-butoxycarbonylamino-6-methyl-heptanoic acid methyl ester 14 (48 mg, 100%) as a colourless oil.

Analytical HPLC Rt=22.65 min (100%); [α]_{D}²³−13.3 (c 0.96 in CH₃OH); δ_{H} (500 MHz; CDCl₃) 0.85 (6H, d, J 6.5, (CH₃)₂CH), 1.16 (2H, m, NHCH(CH₂)₂CH₂), 1.30 (2H, m, NHCHCH₂CH₂), 1.42 (9H, s, C(CH₃)₃), 1.51 (1H, qt, J 7 and 6.5, (CH₃)₃CH), 1.58 (1H, m, NHCHCH_{2A}), 1.74 (1H, m, NHCHCH_{2B}), 3.71 (3H, s, CO₂CH₃), 4.28 (1H, br m, NHCHCO₂CH₃) and 4.99 (1H, br d, J 7.5, NH); δ_{C} (125 MHz; CDCl₃) 22.42 ((CH₃)_{2A}CH), 22.48 ((CH₃)_{2B}CH), 23.01 (NHCHCH₂CH₂), 27.72 ((CH₃)₂CH), 28.27 (C(CH₃)₃), 32.94 (NHCHCH₂), 38.33 (NHCH(CH₂)₂CH₂), 52.13 (CO₂CH₃), 53.39 (NHCHCO₂CH₃), 155.32 (OC(O)NH) and 173.51 (NHCHCO₂CH₃); hrms 296.1836 (MNa.C₁₄H₂₇NO₄Na requires 296.1838 (d 0.7 ppm)); m/z (Electrospray-MS) 274 (53%) and 218 (100%).

c) 2S-2-tert-Butoxycarbonylamino-6-methyl-heptanoic acid:

Following the general procedure for methyl ester saponification, 2S-tert-butoxycarbonylamino-6-methyl-heptanoic acid methyl ester 14 (100 mg, 0.37 mmol) gave 2S-2-tert-butoxycarbonylamino-6-methyl-heptanoic acid (88 mg, 92%) as a solid, and used directly in the subsequent reaction. Analytical 20.04 min (100%); m/z (Electrospray-MS) 260 (8%) and 204 (100%).

d) 2S-2-Amino-6-methyl-heptanoic acid hydrochloride salt:

Following the general procedure of N-Boc removal using 4 M HCl in dioxane, 2S-2-tert-butoxycarbonylamino-6-methyl-heptanoic acid (88 mg, 0.34 mmol) gave 2S-2-amino-6-methyl-heptanoic acid hydrochloride salt (66 mg, 100%) as a solid and used directly in the subsequent reaction; m/z (Electrospray-MS) 160 (100%).

e) 2S-2-(9H-Fluoren-9-ylmethoxycarbonylamino)-6-methyl-heptanoic acid 3:

Following the general procedure for Fmoc protection of an amine 2S-2-amino-6-methyl-heptanoic acid hydrochloride salt (66 mg, 0.34 mmol) gave on purification by flash chromatography over silica gel eluting with CHCl₃/CH₃OH (100:0 to 95:5, v/v), 2S-2-(9H-fluoren-9-ylmethoxycarbonylamino)-6-methyl-heptanoic acid 3 (70 mg, 54%) as amorphous solid, mp 97-98° C.

Analytical HPLC Rt=23.55 min (100%); [α]_{D}²³−14.6 (c 0.74 in CH₃OH); δ_{H} (500 MHz; CDCl₃) 0.84 (6H, d, J 7, (CH₃)₂CH), 1.09 (2H, br m, NHCH(CH₂)₂CH₂), 1.28 (2H, m, NHCHCH₂CH₂), 1.46 (1H, qt, J 7 and 6.5, (CH₃)₃CH), 1.63 (1H, m, NHCHCH_{2A}), 1.84 (1H, m, NHCHCH_{2B}), 4.18 (1H, t, J 7, H-9′), 4.36 (1H, br m, NHCHCO₂H), 4.38 (2H, d, J 6.5, CH₂O), 5.27 (1H, br d, J8, NH), 7.28 (2H, m, H-2′ and H-7′), 7.37 (2H, m, H-3′ and H-6′), 7.57 (2H, m, H-1′ and H-8′) and 7.74 (2H, d, J 7.5, H-4′ and H-5′); δ_{C} (125 MHz; CDCl₃) 22.43 ((CH₃)_{2A}CH), 22.53 ((CH₃)_{2B}CH), 23.04 (NHCHCH₂CH₂), 27.71 ((CH₃)₂CH), 32.44 (NHCHCH₂), 38.29 (NHCH(CH₂)₂CH₂), 47.09 (CH-9′), 53.83 (NHCHCO₂H), 67.05 (CH₂O), 119.95 (CH-4′ and CH-5′), 125.02 (CH-1′ and CH-8′), 127.03 (CH-2′ and CH-7′), 127.68 (CH-3′ and CH-6′), 141.26 (C-4a′ and C-5a′), 143.65 (C-1a′ and C-8a′), 156.10 (OC(O)NH) and 176.90 (NHCHCO₂H); hrms 404.1856 (MNa.C₂₃H₂₇NO₄Na requires 404.1838 (_{d} 4.4 ppm)); m/z (Electrospray-MS) 382 (100%) and 267 (70%).

### EXAMPLE 4

2S,4R-2-(9H-Fluoren-9-ylmethoxycarbonylamino)-4.5-dimethyl-hexanoic acid 5a

a) 2S,4R-2-tert-Butoxycarbonylamino-4,5-dimethyl-hex-5-enoic acid methyl ester 16a; and 2S,4S-2-tert-butoxy-carbonylamino-4,5-dimethyl-hex-5-enoic acid methyl ester 16b:

Following the general procedure for the coupling reaction, toluene-4-sulfonic acid (E)-2-methyl-but-2-enyl ester (0.24 g, 1.00 mmol) was coupled to N-(tert-butoxycarbonyl)-3-iodo-L-alanine methyl ester (247 mg, 0.75 mmol) in the presence of CuBr.SMe₂ (21 mg, 0.10 mmol) to give a residue which was purified by flash chromatography over silica gel, eluting with EtOAc/40:60 petroleum ether (1:9, v/v). Two products were obtained; 2S,4S-2-tert-butoxycarbonylamino-4,5dimethyl-hex-5-enoic acid methyl ester 16a and 2S,4R-2-tert-butoxy-carbonylamino-4,5dimethyl-hex-5-enoic acid methyl ester 16b. ¹H NMR spectroscopy showed that a 1:1 ratio of diastereoisomers was obtained. Compound 16a was tentatively assigned as the anti-isomer on the basis of the chemical shift of the methylene carbon, 110.19, compared with 111.27 for the syn-isomer. These shifts should be compared with shifts of 110.1 and 111.1 reported for the tentatively assigned anti- and syn-N-acetyl analogues.¹⁴ Fractions containing the first eluted component were pooled to give one of the diastereoisomers 16a (65 mg, 32%) as a colourless oil.

Analytical HPLC Rt=22.52 min (90%); [α]_{D}²⁰+12.3 (c 1.06 in CHCl₃);

ₙₘₐₓ(film)/cm⁻¹ 3382 (m), 3070 (m), 2966 (s), 1746 (s), 1716 (s), 1616 (w), 1507 (s) and 886 (m); δ_{H} (500 MHz, CDCl₃) 1.06 (3H, d, J 7, CH₃CH), 1.45 (9H, s, C(CH₃)₃), 1.58 (1H, m, CH₂═C(CH₃)CH), 1.68 (3H, s, CH₂═C(CH₃)), 1.85 (1H, m, NHCHCHCH_{2A}), 1.97 (1H, m, NHCHCH_{2B}), 3.73 (3H, s, CO₂CH₃), 4.29 (1H, m, NHCHCO₂CH₃), 4.72 (1H, s, CH_{2A}═C(CH₃)), 4.95 (1H, d, J 1.5, CH_{2B}═C(CH₃)) and 5.04 (1H, br d, J 7, NH); δ_{C} (125 MHz, CDCl₃) 18.61 (CH₂═C(CH₃)), 21.64 (CH₂═C(CH₃)CH(CH₃)), 28.32 (C(CH₃)₃), 30.79 (CH₂═C(CH₃)CH), 38.06 (NHCHCH₂), 52.00 (NHCHCO₂CH₃), 52.22 (CO₂CH₃), 79.53 (C(CH₃)₃), 110.19 (CH₂═C(CH₃)), 144.62 (CH₂═C(CH₃)), 155.18 (OC(O)NH) and 173.30 (NHCHCO₂CH₃); hrms 294.1684 (MNa.C₁₄H₂₅NO₄ Na requires 294.1681 (d 0.8 ppm)); m/z (Electrospray-MS) 272 (26%) and 216 (100%).

Pooling together the lower eluting component gave the other diastereoisomer 16b (39 mg, 19%) as a colourless oil. Analytical HPLC Rt=22.49 min (95%); [α]_{D}²⁰+16.0 (c 0.60 in CHCl₃); ₙₘₐₓ(film)/cm⁻¹ 3369 (s), 3073 (m), 2969 (s), 1747 (s), 1717 (s), 1617 (w), 1517(s) and 893 (m); 8H (500 MHz, CDCl₃) 1.04 (3H, d, J 7, CH₃CH), 1.44 (9H, s, C(CH₃)₃), 1.55 (1H, m, CH₂═C(CH₃)CH), 1.67 (3H, s, CH₂═C(CH₃)), 1.91 (1H, m, NHCHCH_{2A}), 2.32 (1H, m, NHCHCH_{2B}), 3.72 (3H, s, CO₂CH₃), 4.26 (1H, m, NHCHCO₂CH₃), 4.75 (1H, d, J 1.5, CH_{2A}═C(CH₃)), 4.79 (1H, d, J 1.5, CH_{2B}═C(CH₃)) and 5.46 (1H, br d, J 6, NH); δ_{C} (125 MHz, CDCl₃) 18.51 (CH₂═C(CH₃)), 20.14 (CH₂═C(CH₃)CH(CH₃)), 28.31 (C(CH₃)₃), 30.55 (CH₂═C(CH₃)CH), 37.64 (NHCHCH₂), 52.17 (NHCHCO₂CH₃), 52.22 (CO₂CH₃), 79.74 (C(CH₃)₃), 111.27 (CH₂═C(CH₃)), 147.94 (CH₂═C(CH₃)), 155.36 (OC(O)NH) and 173.83 (NHCHCO₂CH₃); hrms 294.1673 (MNa.C₁₄H₂₅NO₄Na requires 294.1681 (d 2.9 ppm)); m/z (Electrospray-MS) 272 (73%) and 216 (100%).

b) 2S,4R-2-tert-Butoxycarbonylamino-4,5-dimethyl-hexanoic acid methyl ester 17a; and 2S,45-2-tert-butoxycarbonylamino-4,5-dimethyl-hexanoic acid methyl ester 17b:

Following the general procedure for alkene hydrogenation, the first eluted diastereoisomer of 2S,4R-2-tert-butoxycarbonylamino-4,5-dimethyl-hex-5-enoic acid methyl ester 1 6a (63 mg, 0.23 mmol) yielded 2S,4R-2-tert-butoxycarbonylamino-4,5-dimethyl-hexanoic acid methyl ester 17a (60 mg, 95%) as a colourless oil.

Analytical HPLC Rt 22.52 min (90%); [α]_{D}¹⁸+3.3 (c 0.60 in CH₂Cl₂); δ_{H} (500 MHz, CDCl₃) 0.81 (3H, d, J 7, (CH₃)_{2A}CH), 0.84 (3H, d, J 7, (CH₃)₂CHCH(CH₃)), 0.87 (3H, d, J 7, (CH₃)_{2B}CH), 1.10 (1H, m, (CH₃)₂CH), 1.31 (1H, m, (CH₃)₂CHCH(CH₃)), 1.43 (9H, s, C(CH₃)₃), 1.53 (1H, m, NHCHCH_{2A}) 1.75 (1H, m, NHCHCH_{2B}), 3.72 (3H, s, CO₂CH₃), 4.26 (1H, br m, NHCHCO₂CH₃) and 4.96 (1H, br d, J 7, NH); hrms 296.1835 (MNa.C₁₄H₂₇NO₄Na requires 296.1838 (d 1.0 ppm)); m/z (Electrospray-MS) 274 (43%) and 218 (100%).

Following the general procedure for alkene hydrogenation, the second eluted diastereoisomer of 2S,4S-2-tert-butoxycarbonylamino-4,5-dimethyl-hex-5-enoic acid methyl ester 16b (39 mg, 0.14 mmol) yielded 2S,4S-2-tert-butoxycarbonylamino-4,5-dimethyl-hexanoic acid methyl ester 17b (39 mg, 100%) as a coiourless oil.

Analytical HPLC Rt22.49 min (98%); [α]_{D}¹⁸+32.0 (c 0.10 in CH₂Cl₂); δ_{H} (500 MHz, CDCl₃) 0.78 (3H, d, J 7, (CH₃)_{2A}CH), 0.84 (3H, d, J 7, (CH₃)₂CHCH(CH₃)), 0.85 (3H, d, J 7, (CH₃)_{2B}CH), 1.37 (1H, m, NHCHCH_{2A}), 1.43 (9H, s, C(CH₃)₃), 1.52 (1H, m, (CH₃)₂CHCH(CH₃)), 1.64 (1H, m, (CH₃)₂CH), 1.76 (1H, ddd, J 10, 7 and 6, NHCHCH_{2B}), 3.72 (3H, s, CO₂CH₃), 4.29 (1H, br m, NHCHCO₂CH₃) and 4.94 (1H, br d, J 7, NH); δ_{C} (125 MHz, CDCl₃) 15.16 (CH₃)₂CHCH(CH₃)), 17.07 ((CH₃)_{2A}CH), 20.00 ((CH₃)_{2B}CH), 28.26 (C(CH₃)₃), 31.03 (CH₃)₂CHCH(CH₃)), 34.66 (CH₃)₂CHCH(CH₃)), 37.53 (NHCHCH₂), 52.04 (NHCHCO₂CH₃), 52.12 (CO₂CH₃), 79.78 (C(CH₃)₃), 155.17 (OC(O)NH) and 173.89 (NHCHCO₂CH₃); ); hrms 296.1830 (MNa.C₁₄H₂₇NO₄Na requires 296.1838 (d 2.7 ppm)); r/z (Electrospray-MS) 274 (40%) and 218 (100%).

c) 2S,4R-2-tert-Butoxycarbonylamino-4,5-dimethyl-hexanoic acid; and 2S,4S-2-tert-butoxycarbonylamino-4,5-dimethyl-hexanoic acid:

Following the general procedure for methyl ester saponification 2S,4R-2-tert-butoxycarbonylamino-4,5-dimethyl-hexanoic acid methyl ester (60 mg, 0.22 mmol) yielded 2S,4R-2-tert-butoxycarbonylamino-4,5-dimethyl-hexanoic acid (52 mg, 91 %) as a colourless oil and used directly in the subsequent reaction.

Analytical HPLC Rt=20.65 min (100%); m/z (Electrospray-MS) 260 (18%) and 204 (100%).

Following the general procedure for methyl ester saponification 2S,4S-2-tert-butoxycarbonylamino-4,5-dimethyl-hexanoic acid methyl ester (32 mg, 0.12 mmol) yielded 2S,4S-2-tert-butoxycarbonylamino4,5-dimethyl-hexanoic acid (30 mg, 100%) as a colourless oil and used directly in the subsequent reaction. Analytical HPLC Rt =20.45 min (100%); mlz (Electrospray-MS) 260 (20%) and 204 (100%).

d) 2S,4R-2-Amino-4,5-dimethyl-hexanoic acid hydrochloride salt; and 2S,4S-2-amino4,5-dimethyl-hexanoic acid hydrochloride salt:

Following the general procedure of N-Boc removal using 4 M HCl in dioxane, 2S,4R-2-tert-butoxycarbonylamino-4,5-dimethyl-hexanoic acid (52 mg, 0.20 mmol) yielded 2S,4R-2-amino-4,5-dimethyl-hexanoic acid hydrochloride salt (39 mg, 100%) as a solid and used directly in the subsequent reaction; m/z (Electrospray-MS) 160 (76%) and 142 (100%).

Following the general procedure of N-Boc removal using 4 M HCl in dioxane, 2S,4S-2-tert-butoxycarbonylamino-4,5-dimethyl-hexanoic acid (32 mg, 0.12 mmol) yielded 2S,4S-2-amino-4,5-dimethyl-hexanoic acid hydrochloride salt (24 mg, 100%) as a solid and used directly in the subsequent reaction; m/z (Electrospray-MS) 160 (80%) and 142 (100%).

e) 2S,4R-2-(9H-Fluoren-9-ylmethoxycarbonylamino)-4,5-dimethyl-hexanoic acid 5a; and 2S,4S-2-(9H-fluoren-9-ylmethoxycarbonylamino)4,5-dimethyl-hexanoic acid 5b:

Following the general procedure for Fmoc protection of an amine, 2S,4R-2-amino-4,5-dimethyl-hexanoic acid hydrochloride salt (39 mg, 0.20 mmol) gave on purification by flash chromatography over silica gel, eluting with CHCl₃/CH₃OH (100:0 to 95:5, v/v), 2S,4R-2-(9H-fluoren-9-ylmethoxycarbonylamino)-4,5-dimethyl-hexanoic acid 5a (30 mg, 40%) as an amorphous solid, mp 53-54° C. Analytical HPLC Rt 23.46 min (100%); [α]_{D}²³−10.4 (c 1.00 in CH₃OH); δ_{H} (500 MHz, CDCl₃) 0.85 (9H, m, (CH₃)₂CHCH(CH₃)), 1.34 (1H, m, (CH₃)₂CHCH(CH₃)), 1.56 (1H, m, NHCHCH_{2A}), 1.64 (1H, br m, (CH₃)₂CHCH(CH₃), 1.89 (1H, m, NHCHCH_{2B}), 4.21 (1H, t, J 7, H-9′), 4.41 (3H, m, CH₂O and NHCHCO₂H), 5.09 (1H, br d, J 7, NH), 7.29 (2H, m, H-2′ and H-7′), 7.39 (2H, m, H-3′ and H-6′), 7.56 (2H, m H-1′ and H-8′) and 7.76 (2H, d, J 7, H4′ and H-5′); hrms 404.1825 (MNa.C₂₃H₂₇NO₄Na requires 404.1838 (d 3.2 ppm)); m/z (Electrospray-MS) 382 (100%).

### EXAMPLE 5

2S,4S-2-(9H-fluoren-9-ylmethoxycarbonylamino)-4,5-dimethyl-hexanoic acid 5b.

Following the general procedure for Fmoc protection of an amine, 2S,4S-2-amino-4,5-dimethyl-hexanoic acid hydrochloride salt (24 mg, 0.12 mmol) gave on purification by flash chromatography over silica gel, eluting with CHCl₃/CH₃OH (100:0 to 95:5, v/v), 2S,4S-2-(9H-fluoren-9-ylmethoxycarbonylamino)-4,5-dimethyl-hexanoic acid 5b (15 mg, 32%) as an amorphous solid, mp 50-51° C.

Analytical HPLC Rt 23.23 min (100%); [α]_{D}¹⁸−12.8 (c 0.25 in CH₃OH); δ_{H} (500 MHz, CDCl₃) 0.80 (3H, d, J 6.5, (CH₃)_{2A}CH), 0.89 (6H, d, J 6.5, (CH₃)_{2B}CHCH(CH₃)), 1.49 (1H, m, NHCHCH_{2A}), 1.52 (1H, br m, (CH₃)₂CHCH(CH₃)), 1.66 (1H, br m, (CH₃)₂CHCH(CH₃)), 1.91 (1H, br m, NHCHCH_{2B}), 4.22 (1H, t, J 7, H-9′), 4.42 (3H, m, CH₂O and NHCHCO₂H), 5.13 (1H, br d, J 7, NH), 7.32 (2H, m, H-2′ and H-7′), 7.39 (2H, m, H-3′ and H-6′), 7.56 (2H, m, H-1′ and H-8′) and 7.76 (2H, d, J 7, H-4′ and H-5′); dC (125 MHz; CDCl₃) 15.08 ((CH₃)₂CHCH(CH₃)), 16.94 ((CH₃)_{2A}CH), 20.10 ((CH₃)_{2B}CH), 30.94 ((CH₃)₂CHCH(CH₃)), 34.73 ((CH₃)₂CHCH(CH₃)), 37.13 (NHCHCH₂), 47.13 (CH-9′), 52.30 (NHCHCO₂H), 66.79 (CH₂O), 119.70 (CH-4′ and CH-5′), 124.78 (CH-1′ and CH-8′), 126.79 (CH-2′ and CH-7′), 127.44 (CH-3′ and CH-6′), 141.05 ( C-4a′ and C-5a′), 143.61 (C-1a′ and C-8a′), 155.68 (OC(O)NH) and 178.00 (NHCHCO₂H); hrms 404.1841 (MNa.C₂₃H₂₇NO₄Na requires 404.1838 (d 0.7 ppm)); m/z (Electrospray-MS) 382 (100%).

### EXAMPLE 6

a) 2S-2-tert-butyloxycarbonylamino-5,6-dimethyl-hept-5-enoic methyl ester 18; and 2S-2-tert-butyloxycarbonylamino-4,4,5-trimethyl-hex-5-enoic methyl ester 19:

Following the general procedure for zinc coupling reactions, 1-bromo-2,3-dimethylbut-2-ene (5.45 g, 33.46 mmol) was coupled to N-(tert-butoxycarbonyl)-3-iodo-L-alanine methyl ester (10.00 g, 30.40 mmol) in the presence of CuBr.SMe₂ (0.80 g, 3.89 mmol) to give a residue which on purification by flash chromatography over silica gel eluting with EtOAc/heptane (1:9, v/v) gave two regioisomers in a ratio of 1:1 as established by ¹H NMR spectroscopy. The first eluted component was 2S-2-tert-butyloxycarbonylamino-5,6-dimethyl-hept-5-enoic methyl ester and further elution afforded 2S-2-tert-butyloxycarbonylamino-4,4,5-trimethyl-hex-5-enoic methyl ester. Fractions containing the initial component were pooled and reduced in vacuo to give 2S-2-tert-butyloxycarbonylamino-5,6-dimethyl-hept-5-enoic methyl ester 18 (2.51 g, 29%)-as a colourless oil.

Analytical HPLC Rt=21.96 min (100%); [α]_{D}²²+26.1 (c 1.02 in CH₂Cl₂);

(Found: C, 63.1; H, 9.3; N, 4.9. C₁₅H₂₇NO₄ requires C, 63.1; H. 9.5; N, 4.9%);

ₙₘₐₓ(film)/cm⁻¹ 3366 (m), 3154 (m), 2978 (s), 1744 (s), 1718 (s), 1506 (s), 1366 (s) and 1164 (s); 5H (500 MHz, CDCl₃) 1.43 (9H, s, C(CH₃)₃), 1.60 (9H, m, (CH₃)₂C═C(CH₃)), 1.66 (1H, m, NHCHCH_{2A}), 1.85 (1H, m, NHCHCH_{2B}), 2.00 (1H, ddd, J 13, 12.5 and 5, NHCHCH₂CH_{2A}), 2.07 (1H, ddd, J 13, 10.5 and 6, NHCHCH₂CH_{2B}), 3.72 (3H, s, CO₂CH₃), 4.25 (1H., br m, NHCHCO₂CH₃) and 5.00 (1H, br d, J 7, NH); δ_{C} (125 MHz, CDCl₃) 18.14 ((CH₃)₂C═C(CH₃)), 19.92 ((CH₃)_{2A}C═C(CH₃)), 20.53 ((CH₃)₂₈C═C(CH₃)), 28.26 (C(CH₃)₃), 30.01 (NHCHCH₂CH₂), 30.86 (NHCHCH₂), 52.10 (OCH₃), 53.41 (NHCHCO₂CH₃), 79.74 (C(CH₃)₃), 125.36 ((CH₃)₂C═C(CH₃)), 125.93 ((CH₃)₂C═C(CH₃), 155.30 (OC(O)NH) and 173.34 (NHCHCO₂CH₃); hrms 308.1829 (MNa. C₁₅H₂₇NO₄Na requires 308.1838 (d 2.9 ppm)); m/z (Electrospray-MS) 286 (100%).

Pooling together the lower eluting component gave 2S-2-tert-butyloxycarbonylamino-4,4,5-trimethyl-hex-5-enoic methyl ester 19 (2.60 g, 30%) as a colourless oil.

Analytical HPLC Rt=21.02 min (100%); [α]_{D}¹⁸+3.5 (c 0.83 in CH₂Cl₂);

(Found: C, 62.7; H, 9.3; N, 4.95. C₁₅H₂₇NO₄ requires C, 63.1; H, 9.5; N, 4.9%); ₙₘₐₓ(film)/cm⁻¹ 3368 (s), 3091 (m), 2934 (s), 1748 (s), 1717 (s) and 1516(s); δ_{H} (500 MHz, CDCl₃) 1.08 (3H, s, CH₂═C(CH₃)C(CH₃)_{2A}), 1.10 (3H, s, CH₂═C(CH₃)C(CH₃)_{2B}), 1.40 (9H, s, C(CH₃)₃), 1.59 (1H, dd, J 14.5 and 9, NHCHCH_{2A}), 1.73 (3H, d, J 1, H₂C═C(CH₃)), 1.90 (1H, dd, J 14.5 and 4, NHCHCH_{2B}), 3.67 (3H, s, CO₂CH₃), 4.22 (1H, br m, NHCHCO₂CH₃), 4.77 (1H, d, J 1, CH_{2A}═C(CH₃)) and 4.81 (2H, br m, CH_{2B}═C(CH₃) and NH); δ_{C} (125 MHz, CDCl₃) 19.31 (CH₂═C(CH₃)), 27.13-(CH₂═CC(CH₃)C(CH₃)_{2A}), 27.54 (CH₂═CC(CH₃)C(CH₃)_{2B}), 28.28 C(CH₃)₃), 38.45 ((CH₂═C(CH₃)C(CH₃)₂), 42.91 (NHCHCH₂), 51.29 (NHCHCO₂CH₃), 52.04 (CO₂CH₃), 79.64 (C(CH₃)₃), 110.88 (CH₂=C(CH₃)), 150.57 (CH₂═C(CH₃)), 154.96 (OC(O)NH) and 174.04 (NHCHCO₂CH₃); hrms 308.1838 (MNa.C₁₅H₂₇NO₄Na requires 308.1838 (d 2.2 ppm)); m/z (Electrospray-MS) 286 (100%).

b) 2S,5S-2-tert-Butoxycarbonylamino-5,6-dimethyl-heptanoic acid methyl ester; and 2 S,5R-2-tert-butoxycarbonylamino-5,6-dimethyl-heptanoic acid methyl ester 20:

Following the general procedure for alkene hydrogenation, 2S-2-tert-butyloxycarbonylamino-5,6-dimethyl-hept-5-enoic methyl ester 18 (6.78 g, 23.79 mmol) yielded on purification by flash column chromatography over silica gel, eluting with EtOAc/heptane (1:9, v/v), an inseparable mixture of 2S,5S-2-tert-butoxycarbonylamino-5,6-dimethyl-heptanoic acid methyl ester and 2S,5R-2-tert-butoxycarbonylamino-5,6-dimethyl-heptanoic acid methyl ester 20 (6.63 g, 97%) as a colourless oil.

Analytical HPLC Rt=24.06 min (100%); [α]_{D}²³−12.1 (c 1.26 in CH₃OH);

(Found: C, 62.9; H, 10.1; N, 4.9. C₁₅H₂₉NO₄Na requires C, 62.7; H, 10.2 and N, 4.9%); δ_{H} (500 MHz, CDCl₃) 0.76 (3H, dd, J 7 and 3.5, (CH₃)₂CHCH(CH₃)), 0.78 (3H, dd, J 7 and 1.5, (CH₃)_{2A}CHCH(CH₃)), 0.83 ((3H, dd, J 7 and 1.5, (CH₃)_{2B}CHCH(CH₃)), 1.09 (1H, m, NHCHCH₂CH_{2A}), 1.26 (1H, m, (CH₃)₂CHCH(CH₃)), 1.37 (1H, m, NHCHCH₂CH_{2B}), 1.42 (9H, s, C(CH₃)₃), 1.53 (1.5H, m, (CH₃)₂CHCH(CH₃) and 0.5 NHCHCH_{2A}), 1.63 (0.5H, m, 0.5 NHCHCH_{2A}), 1.74 (0.5H, br m, 0.5 NHCHCH_{2B}), 1.84 (0.5H, br m, 0.5 NHCHCH_{2B}), 3.72 (3H, s, CO₂CH₃), 4.25 (1H, br m, NHCHCO₂CH₃) and 4.99 (1H, br m, NH); δ_{C} (125 MHz, CDCl₃) 15.16 and 15.18 ((CH₃)₂CHCH(CH₃)), 17.78 and 17.91 ((CH₃)_{2A}CHCH(CH₃)), 20.06 and 20.14 ((CH₃)_{2B}CHCH(CH₃)), 28.26 (C(CH₃)₃), 29.38 and 29.47 (NHCHCH₂CH₂), 30.60 and 30.75 (NHCHCH₂), 31.66 and 31.83 ((CH₃)₂CHCH(CH₃)), 38.07 and 38.27 ((CH₃)₂CHCH(CH₃)), 52.10 (NHCHCO₂CH₃), 53.55 and 53.68 (NHCHCO₂CH₃), 79.75 (C(CH₃)₃), 155.306 (OC(O)NH) and 173.43 and 173.49 (NHCHCO₂CH₃); hrms 310.1982 (MNa.C₁₅H₂₉NO₄Na requires 310.1994 (d 4.1 ppm)); m/z (Electrospray-MS) 288 (68%) and 232 (74%).

c) 2S,5S-2-tert-Butoxycarbonylamino-5,6-dimethyl-heptanoic acid; and 2S,5R-2-tert-butoxycarbonylamino-5,6-dimethyl-heptanoic acid 22:

Following the general procedure for methyl ester saponification, 2S,5S-2-tert-butoxycarbonylamino-5,6-dimethyl-heptanoic acid methyl ester and 2S,5R-2-tert-butoxycarbonylamino-5,6-dimethyl-heptanoic acid methyl ester 20 (6.60 g, 23.00 mmol) gave after purification by flash chromatography over silica gel, eluting with CHCl₃/MeOH (95:5, v/v), 2S,5S-2-tert-butoxycarbonylamino-5,6-dimethyl-heptanoic acid and 2S,5R-2-tert-butoxycarbonylamino-5,6-dimethyl-heptanoic acid 22 (6.28 g, 100%) as a colourless oil.

Analytical HPLC Rt=21.44 min (100%); δ_{H} (500 MHz, CDCl₃) 0.79 (6H, d, J 6.5, (CH₃)_{2A}CHCH(CH₃)), 0.84 (3H, d, J 7, (CH₃)_{2B}CHCH(CH₃)), 1.15 (1H, m, NHCHCH₂CH_{2B}), 1.28 (1H, m, (CH₃)₂CHCH(CH₃)), 1.40 (1H, m, NHCHCH₂CH_{2B}), 1.44 (9H, s, C(CH₃)₃), 1.54 (1.5H, br m, (CH₃)₂CHCH(CH₃) and 0.5 NHCHCH_{2A}), 1.68 (0.5H, br m, 0.5 NHCHCH_{2A}), 1.79 (0.5H, br m, 0.5 NHCHCH_{2B}), 1.89 (0.5H, br m, 0.5 NHCHCH_{2B}), 4.25 (1H, br m, NHCHCO₂CH₃) and 5.09 (1H, br s, NH), δ_{C} (125 MHz, CDCl₃) 15.12 ((CH₃)₂CHCH(CH₃)), 17.75 and 17.89 ((CH₃)_{2A}CHCH(CH₃)), 20.12 and 20.23 ((CH₃)_{2B}CHCH(CH₃)), 28.27 (C(CH₃)₃), 29.46 and 29.62 (NHCHCH₂CH₂), 30.30 and 30.48 (NHCHCH₂), 31.66 and 31.83 ((CH₃)₂CHCH(CH₃)), 38.09 and 38.34 ((CH₃)₂CHCH(CH₃)), 53.81 and 53.99 (NHCHCO₂CH₃), 80.01 (C(CH₃)₃), 155.69 (OC(O)NH) and 177.61 (NHCHCO₂H); hrms 296.1831 (MNa. C₁₄H₂₇NO₄Na requires 296.1838 (d 2.4 ppm)); m/z (Electrospray-MS) 274 (19%) and 218 (100%).

d) 2S,5S-2-Amino-5,6-dimethyl-heptanoic acid hydrochloride salt; and 2S, 5R-2-amino-5,6-dimethyl-heptanoic acid hydrochloride salt.

Following the general procedure of N-Boc removal using 4 M HCl in dioxane, 2S,5S-2-tert-butoxycarbonylamino-5,6-dimethyl-heptanoic acid and 2S,5R-2-tert-butoxycarbonylamino-5,6-dimethyl-heptanoic acid (2.47 g, 9.05 mmol) gave 2S,55-2-amino-5,6-dimethyl-heptanoic acid hydrochloride salt and 2S,5R-2-amino-5,6-dimethyl-heptanoic acid hydrochloride salt (1.84 g, 97%) as a solid and used in the subsequent reaction without further purification; m/z (Electrospray-MS) 174 (100%).

e) 2S,5S-2-(9H-Fluoren-9-ylmethoxycarbonylamino)-5,6-dimethyl-heptanoic acid; and 2S,5R-2-(9H-Fluoren-9-ylmethoxycarbonylamino)-5,6-dimethyl-heptanoic acid 6.

Following the general procedure for Fmoc protection of an amine, 2S,5S-2-amino-5,6-dimethyl-heptanoic acid hydrochloride salt and 2S,5R-2-amino-5,6-dimethyl-heptanoic acid hydrochloride salt (1.84 g, 8.78 mmol) gave on purification by flash chromatography over silica gel eluting with CHCl₃/CH₃OH (100:0 to 95:5, v/v), 2S,5S-2-(9H-fluoren-9-ylmethoxycarbonylamino)-5,6-dimethyl-heptanoic acid and 2S,5R-2-(9H-fluoren-9-ylmethoxycarbonylamino)-5,6-dimethyl-heptanoic acid 6 (1.94 g, 56%) as an amorphous solid, mp 43-44° C.

Analytical HPLC Rt=24.52 min (100%); dH (500 MHz; CDCl₃) 0.78 (6H, m, (CH₃)_{2A}CHCH(CH₃)), 0.84 (3H, d, J 6.5, (CH₃)_{2B}CHCH(CH₃)), 1.15 (1H, m, NHCHCH₂CH_{2A}), 1.29 (1H, m, (CH₃)₂CHCH(CH₃)), 1.40 (1H, m, NHCHCH₂CH_{2B}), 1.54 (1H, m, (CH₃)₂CHCH(CH₃)), 1.64 (0.5H, m, 0.5 NHCHCH_{2A}), 1.73 (0.5H, m, 0.5 NHCHCH_{2A}), 1.85 (0.5H, m, 0.5 NHCHCH_{2B}), 1.94 (0.5H, m, 0.5 NHCHCH_{2B}), 4.22 (1H, t, J 7, H-9′), 4.37 (1H, m, NHCHCO₂H), 4.41 (2H, br d, J 7, CH₂O), 5.29 (1H, br s, NH), 7.27 (2H, m, H-2′ and H-7′), 7.37 (2H, m, H-3′ and H-6′), 7.56 (2H, m, H-1′ and H-8′) and 7.75 (2H, d, J 7, H-4′ and H-5′); dC (125 MHz; CDCl₃) 15.12 ((CH₃)₂CHCH(CH₃)), 17.70 and 17.94 ((CH₃)_{2A}CHCH(CH₃)), 20.14 and 20.25 ((CH₃)_{2B}CHCH(CH₃)), 29.44 and 29.58 (NHCHCH₂CH₂), 30.26 and 30.39 (NHCHCH₂), 31.59 and 31.86 ((CH₃)₂CHCH(CH₃)), 38.10 and 38.28 ((CH₃)₂CHCH(CH₃)), 47.11 (CH-9′), 53.98 and 54.08 (NHCHCO₂H), 67.08 and 67.61 (CH₂O), 119.72 (CH-4′ and CH-5′), 124.80 (CH-¹′ and CH-8′), 126.81 (CH-2′ and CH-7′), 127.46 (CH-3′ and CH-6′), 141.05 ( C-4a′ and C-5a′), 143.47 (C-1a′ and C-8a′), 155.89 (OC(O)NH) and 177.19 (NHCHCO₂H); hrms 418.1992 (MNa.C₂₄H₂₉NO₄Na requires 418.1994 (d 0.62 ppm)); m/z (Electrospray-MS) 396 (46%) and 267 (100%).

### EXAMPLE 7

2S-(9H-Fluoren-9-ylmethoxycarbonylamino)-4,4,5-trimethyl-hexanoic acid 7

a) 2S-2-tert-Butoxycarbonylamino-4,4,5-trimethyl-hexanoic acid methyl ester 21:

Following the general procedure for alkene hydrogenation, 2S-2-tert-butyloxycarbonylamino-4,4,5-trimethyl-hex-5-enoic methyl ester 19 (5.85 g, 3.51 mmol) yielded on purification by flash column chromatography over silica gel, eluting with EtOAc/heptane (1:5, v/v), 2S-2-tert-butoxycarbonylamino-4,4,5-trimethyl-hexanoic acid methyl ester 21 (5.60 g, 95%) as a colourless oil.

Analytical HPLC Rt=22.91 min (100%); [α]_{D}¹⁷−5.7 (c 0.83 in CH₂Cl₂);

(Found: C, 62.7; H, 10.0; N, 4.8. C₁₅H₂₉NO₄ requires C, 62.7; H, 10.2; N, 4.9%); δ_{H} (500 MHz, CDCl₃) 0.83 (3H, d, J 7, (CH₃)_{2A}CHC(CH₃)₂), 0.84 (3H, d, J 7, (CH₃)_{2B}CHC(CH₃)₂), 0.85 (3H, S, (CH₃)₂CHC(CH₃)_{2A}), 0.89 (3H, s, (CH₃)₂CHC(CH₃)_{2B}), 1.40 (1H, dd, J 14.5 and 9, NHCHCH_{2A}), 1.42 (9H, s, C(CH₃)₃), 1.54 (1H, q, J 7, (CH₃)₂CH), 1.72 (1H, dd, J 14.5 and 3, NHCHCH_{2B}), 3.71 (3H, s, CO₂CH₃), 4.34 (1H, br m, NHCHCO₂CH₃) and 4.79 (1H, br d, J 8, NH); δ_{C} (125 MHz, CDCl₃) 17.22 ((CH₃)_{2A}CHC(CH₃)₂), 17.34 ((CH₃)_{2B}CHC(CH₃)₂), 23.81 ((CH₃)₂CHC(CH₃)_{2A}), 24.41 ((CH₃)₂CHC(CH₃)_{2B}), 28.28 (C(CH₃)₃), 35.33 ((CH₃)₂CHC(CH₃)₂), 35.94 ((CH₃)₂CHC(CH₃)₂), 42.67 (NHCHCH₂), 50.69 (NHCHCO₂CH₃), 52.14 (CO₂CH₃), 79.80 (C(CH₃)₃), 155.08 (OC(O)NH) and 174.57 (NHCHCO₂CH₃); hrms 310.1987 (MNa C₁₅H₂₉NO₄Na requires 310.1994 (d 2.4 ppm)); m/z (Electrospray-MS) 288 (48%) and 232 (100%).

b) 2S-2-tert-Butoxycarbonylamino-4,4,5-trimethyl-hexanoic acid 23:

Following the general procedure for methyl ester saponification, 2S-2-tert-butoxycarbonylamino-4,4,5-trimethyl-hexanoic acid methyl ester 21 (5.60 g, 19.49 mmol) gave on purification by flash column chromatography over silica gel, eluting with CHCl₃/CH₃OH (95:5, v/v), 2S-2-tert-butoxycarbonylamino-4,4,5-trimethyl-hexanoic acid 23 (5.33 g, 100%) as a colourless oil.

Analytical HPLC Rt=22.91 min (100%); [α]_{D}¹⁷−19.1 (c 0.70 in CH₂Cl₂); δ_{H} (500 MHz, CDCl₃) 0.83 (6H, d, J 7, (CH₃)₂CHC(CH₃)₂), 0.86 (3H, s, (CH₃)₂CHC(CH₃)_{2A}), 0.90 (3H, s, (CH₃)₂CHC(CH₃)_{2B}), 1.42 (9H, s, C(CH₃)₃), 1.43 (1H, m, NHCHCH_{2A}), 1.55 (1H, m, (CH₃)₂CH), 1.82 (1H, br d, J 14.5, NHCHCH_{2B}), 4.31 (1H, br m, NHCHCO₂CH₃) and 4.86 (1H, br d, J 8, NH); δ_{C} (125 MHz, CDCl₃) 17.23 ((CH₃)_{2A}CHC(CH₃)₂), 17.36 ((CH₃)_{2B}CHC(CH₃)₂), 23.82 ((CH₃)₂CHC(CH₃)_{2A}), 24.44 ((CH₃)₂CHC(CH₃)_{2B}), 28.30 (C(CH₃)₃ ), 35.41 (23.81 ((CH₃)₂CHC(CH₃)₂), 35.99 ((CH₃)₂CHC(CH₃)₂), 42.42 (NHCHCH₂), 50.84 (NHCHCO₂CH₃), 80.12 (C(CH₃)₃, 155.44 (OC(O)NH) and 178.93 (NHCHCO₂H); hrms 296.1826 (MNa. C₁₄H₂₇NO₄Na requires 296.1838 (d 4.1 ppm)); m/z (Electrospray-MS) 274 (38%) and 218 (100%).

c) 2S-2-Amino-4,4,5-trimethyl-hexanoic acid hydrochloride salt:

Following the general procedure of N-Boc removal using 4 M HCl in dioxane, 2S-2-tert-butoxycarbonylamino-4,4,5-trimethyl-hexanoic acid (1.85 g, 6.80 mmol) gave 2S-2-amino-4,4,5-trimethyl-hexanoic acid hydrochloride salt (1.42 g, 100%) as a solid; m/z (Electrospray-MS) 174 (100%).

d) 2S-(9H-Fluoren-9-ylmethoxycarbonylamino)-4,4,5-trimethyl-hexanoic acid 7:

Following the general procedure for Fmoc protection of an amine, 2S-2-amino-4,4,5-trimethyl-hexanoic acid hydrochloride salt (1.42 g, 6.78 mmol) gave on purification by flash chromatography over silica gel eluting with CHCl₃/CH₃OH (100:0 to 95:5, v/v), 2S9H-fluoren-9-ylmethoxycarbonylamino)-4,4,5-trimethyl-hexanoic acid 7 (1.23 g, 46%) as an amorphous solid, mp 61-62° C.

Analytical HPLC Rt=24.28 min (100%); [α]_{D}¹⁷−15.0 (c 0.62 in CH₂Cl₂); _{dH} (500 MHz; CDCl₃) 0.85 (9H, m, (CH₃)₂CHC(CH₃)_{2A}), 0.91 (3H, s, (CH₃)₂CHC(CH₃)_{2B}), 1.46 (1H, dd, J 14 and 9, NHCH_{2A}), 1.54 (1H, m, (CH₃)₂CH), 1.88 (1H, dd, J 14 and 3, NHCH_{2B}), 4.21 (1H, t, J 6.5, H-9′), 4.40 (3H, br m, NHCHCO₂H and CH₂O), 5.10 (1H, br d, J 7.5, NH), 7.27 (2H, m, H-2′ and H-7′), 7.36 (2H, m, H-3′ and H-6′), 7.57 (2H, m, H-1′ and H-8′) and 7.74 (2H, d, J 7, H-4′ and H-5′); dC (125 MHz; CDCl₃) 17.01 ((CH₃)_{2A}CH), 17.16 ((CH₃)_{2B}CH), 23.69 ((CH₃)₂CHC(CH₃)_{2A}), 24.27 ((CH₃)₂CHC(CH₃)_{2A}), 35.27 ((CH₃)₂CHC(CH₃)₂), 35.73 ((CH₃)₂CH), 41.88 (NHCHCH₂), 46.93 (CH-9′), 54.20 (NHCHCO₂H), 66.79 (CH₂O), 119.70 (CH-4′ and CH-5′), 124.78 (CH-1′ and CH-8′), 126.79 (CH-2′ and CH-7′), 127.44 (CH-3′ and CH-6′), 141.05 (C-4a′ and C-5a′), 143.61 (C-1a′ and C-8a′), 155.68 (OC(O)NH) and 178.00 (NHCHCO₂H); hrms 418.1990 (MNa.C₂₄H₂₉NO₄Na requires 418.1994 (_{d} 1.1 ppm)); m/z (Electrospray-MS) 396 (100%).

### REFERENCES

1 R. F. W. Jackson, N. Wishart, A. Wood, K. James, and M. J. Wythes, J. Org. Chem., 1992, 57, 3397.

2 M. J. Dunn, R. F. W. Jackson, J. Pietruszka, and D. Turner, J. Org. Chem., 1995, 60, 2210.

3 R. F. W. Jackson, R. J. Moore, C. S. Dexter, J. Elliott, and C. E. Mowbray, J. Org. Chem., 1998, 63, 7875.

4 C. S. Dexter and R. F. W. Jackson, J. Chem. Soc., Chem. Commun., 1998, 75.

5 J. Shoji and R. Sakazaki, J. Antibiotics, 1970, 23, 519.

6 T. Shiba, Y. Mukunoki, and H. Akiyama, Bull. Chem. Soc. Jpn., 1975, 48, 1902.

7 W. F. J. Karstens, M. Stol, F. Rutjes, and H. Hiemstra, Synlett, 1998, 1126.

8 W. F. J. Karstens, M. J. Moolenaar, F. Rutjes, U. Grabowska, W. N. Speckamp, and H. Hiemstra, Tetrahedron Lett., 1999, 40, 8629.

9 L. A. Paquette and G. D. Maynard, J. Am. Chem. Soc., 1992, 114, 5018.

10 M. A. Umbreit and K. B. Sharpless, Org. Synth., 1981, 60, 29.

11 M. L. Hill and R. A. Raphael, Tetrahedron, 1990, 46, 4587.

12 W. J. E. Parr, J. Chem. Res. (S), 1981, 354.

13 M. J. Kurth and H. W. Decker, J. Org. Chem., 1985, 50, 5769.

14 J. V. Duncia, P. T. Lansbury, T. Miller, and B. B. Snider, J. Am. Chem. Soc., 1982, 104, 1930.

15 J. J. McCullough, W. K. Macinnis, C. J. L. Lock, and R. Faggiani, J. Am. Chem. Soc., 1982, 104, 4644.

1. A compound of the formula I: 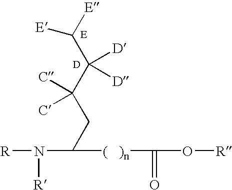
wherein
R is H or an amine protecting group;
R′ is H, C₁-C₆ alkyl, C₂-C₆ alkenyl, ArC₀-C₆ alkyl or HetC₀-C₆ alkyl,
R″ is H or a carboxy protecting group;
( ) is a methylene group;
n is 0, 1 or 2;
C′, C″, D′, E′ and E′ are hydrogen (h) or a group selected from C₁-C₆ alkyl, C₂-C₆ alkenyl, ArC₀-C₆ alkyl or HetC₀-c₆ alkyl, (“Alk”)
D″ is H or an unsaturation between carbon atom D and carbon atom E in the following permutations:

| C′ | C″ | D′ | D″ | E′ | E″ |
|---|---|---|---|---|---|
| H | H | H | H | Alk | Alk |
| H | H | H | ene | Alk | Alk |
| H | H | Alk | H | Alk | Alk |
| H | H | Alk | ene | Alk | Alk |
| H | Alk | Alk | H | H | H |
| H | Alk | Alk | ene | H | H |
| Alk | Alk | H | H | H | H |
| Alk | Alk | H | ene | H | H |
| Alk | Alk | Alk | H | H | H |
| Alk | Alk | Alk | ene | H | H |

wherein the stereochemistry at the alpha carbon is at least 85% enantiomerically pure;
with the proviso that R, R′ and R″ are not all H when C′, C″ and D′ are all H and E′ and E″ are both methyl.

2. A compound according to claim 1, wherein the stereochemic configuration at the alpha carbon defines an L-amino acid.

3. A compound according to any preceding claim, wherein R″ is H.

4. A compound according to any preceding claim wherein R″ is H and R″ is an amine protecting group.

5. A compound according to claim 4, wherein the amine protecting group is selected from Fmoc, Troc, Boc and Cbz.

6. A compound according to claim 5, wherein the protecting group is Fmoc.

7. A compound according to any preceding claim wherein C′, C″ and D′ are hydrogen and E′ and E″ are independently Alk.

8. A compound according to claim 7, wherein E and E″ are methyl.

9. A compound according to any of claims 1-6, wherein C′ and C″ are hydrogen and D′, E″ and E″ are Alk.

10. A compound according to claim 9 wherein D′, E′ and E″ are methyl.

11. A compound according to any of claims 1-6 wherein C′ is hydrogen, C″ is Alk and the intervening carbon has the (R) stereochemistry.

12. A compound according to any of claims 1-6, wherein C′ is hydrogen and C″ is Alk and the intervening carbon has the (S) stereochemistry.

13. A compound according to claim 11 or 12 wherein C″ is methyl.

14. A compound according to claim 11, 12 or 13 wherein D′ is Alk and E′ and E″ are hydrogen.

15. A compound according to claim 13 wherein D′ is methyl.

16. A compound according to any of claims 1-6, wherein C′ and C″ are Alk and D′, E′ and E″ are hydrogen.

17. A compound according to claim 16, wherein C′ and C″ are methyl.

18. A compound according to any of claims 1-6, wherein C′, C″ and D′ are Alk and E′ is hydrogen.

19. A compound according to any preceding claim wherein n is 0, that is ( ) is a bond.

20. Use of a compound as defined in any preceding claim in the synthesis of a peptide or peptidomimetic.

21. Use according to claim 20 wherein the peptidomimetic is a protease inhibitor.

22. A method of synthesising a compound of the formula I 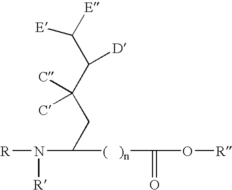
wherein
R are independently H or an amine protecting group;
R′ is C₁-C₆ alkyl, C₂-C₆ alkenyl, ArC₀-C₆ alkyl or HetC₀-C₆ alkyl,
R″ is H or a carboxy protecting group;
( ) is a methylene group;
n is 0, 1 or 2;
C′, C″, D′, E′ and E′ are hydrogen (H) or a group selected from C₁-C₆ alkyl, C₂-C₆ alkenyl, ArC₀-C₆ alkyl or HetC₀-C₆ alkyl, (“Alk”) in the following permutations:

| C′ | C″ | D′ | E′ | E″ |
|---|---|---|---|---|
| H | H | H | Alk | Alk |
| H | H | Alk | Alk | Alk |
| H | Alk | Alk | H | H |
| Alk | Alk | H | H | H |
| Alk | Alk | Alk | H | H |
| Alk | H | H | H | H |

comprising the steps of reacting a zinc reagent of the formula: 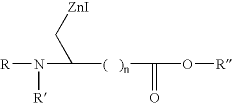
wherein R is an amine protecting group, R′ is H, C₁-C₆ alkyl, C2-C6 alkenyl, ArC₀-C₆ alkyl or HetC₀-C₆ alkyl, and R′ is a carboxy protecting group, with an allylic electrophile; separation of isomers, hydrogenation of the double bond and deprotection as necessary.

22. A method according to claim 21, wherein the zinc reagent is derived from L-serine.

23. A method according to claim 21 or 22, wherein the separation comprises a selective epoxidation of a compound of the formula: 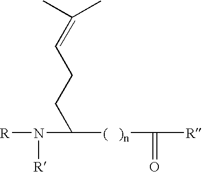
where R, R′, R″, ( ) and n are as defined in claim 20.

24. A method according to any of claims 21-23, wherein the reaction further comprises a catalytic amount of CuBr.DMS.

25. A method according to any of claims 21-24, further comprising replacement of the amine and/or carboxy protecting group with a further protecting group.

26. A method according to claim 25, wherein the replacement comprises deprotection of the carboxy protecting group whereby R″ becomes hydrogen and replacement of the amino protecting group whereby R becomes Fmoc.
